(19) United States
(12) Patent Application Publication
(10) Pub. No.: US 2007/0172837 A1
(43) Pub. Date: Jul. 26, 2007

(54) Flow cell comprising ports and methods for using the same

(76) Inventors: Kyle Schleifer, Mountain View, CO (US); Bill James Peck, Mountain View, CO (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/338,283

(22) Filed: Jan. 23, 2006

(51) Int. Cl. C12Q 1/68; C12M 1/34

(52) U.S. Cl. 435/6; 435/287.2; 427/2.11

(57) ABSTRACT

A flow cell having a plurality of independently controllable inlet and/or outlet ports is provided. In certain embodiments, a subject flow cell contains: a) a chamber having a plurality of independently controllable inlet ports and a plurality of independently controllable outlet ports; b) a first manifold in fluid communication with the inlet ports; and c) a second manifold in fluid communication with the outlet ports. Exemplary methods of using a subject flow cell to control liquid flow over a planar substrate are provided.

### BACKGROUND

Flow cells are employed in a variety of diagnostic and chemical synthesis methods. For example, flow cells are employed in the fabrication of chemical arrays, and for contacting a sample with a planar substrate in a variety of diagnostic methods. In one use, flow cells are employed to make and use chemical arrays, e.g. an array of biopolymers (e.g., polynucleotides or polypeptides) that may be employed in binding assays to identify analytes in a sample. In one use, a monomer for a biopolymer is deposited onto a particular position upon a surface of a planar substrate, and, after deposition of the monomer, the planar substrate is placed in a flow cell where the surface of the substrate is contacted with reactants that chemically modify the deposited monomer. In another example, a chemical array made by any method may be contacted with a sample containing labeled analytes in a flow cell. After contact with the sample and without removing the substrate from the flow cell, the chemical array may be washed to remove unbound analytes. Analytes that are bound to the substrate may be detected by detecting label associated with the substrate.

Despite the widespread use of flow cells in a variety of important applications, flow of liquid through flow cells is quite inefficient in that liquid introduced into a flow cell generally does not make uniform contact with the substrate disposed in the flow cell. For example, in conventional flow cells, there are so-called “dead spots” over which introduced liquid may not flow. Non-uniform contact between a liquid and a substrate in a flow cell may lead to undesirable side reactions, reduced reaction yields, and, if the substrate is to be employed in diagnostic applications, reduced sensitivity.

Thus, there is a need for improved flow cells in which liquid makes uniform contact with the surface of a planar substrate placed in a flow cell. The invention described herein meets this need, and others.

Literature of interest includes: published U.S. patent application Ser. Nos. 20030003222, 20030003504, 20030112022, 200030228422, 200030232123 and 20030232140.

### SUMMARY

In one embodiment, the invention provides a flow cell having a plurality of controllable inlet and/or outlet ports. In one aspect, the subject flow cell contains a first manifold in fluid communication with the inlet ports; and c) a second manifold in fluid communication with the outlet ports. In one embodiment, a subject flow cell may be employed in conjunction with a pulse-jet fluid deposition device as part of an apparatus for fabrication of a chemical array. Exemplary methods of using a subject flow cell to control liquid flow over a planar substrate are provided.

In certain embodiments, a flow cell comprising: a) a chamber comprising: i. a set of inlet ports and ii. a set of outlet ports; b) a first manifold in fluid communication with the inlet ports; and c) a second manifold in fluid communication with the outlet ports, wherein a plurality of said set of inlet ports and/or said set of outlet ports is independently controllable, is provided.

In certain embodiments, the inlet ports and said outlet ports may be connected to the first and second manifolds via independently actuatable valves (e.g., solenoid valves).

In certain embodiments, the flow into and out of the first manifold and/or the second manifold is independently controllable.

In particular embodiments, the flow cell may be adapted for contacting a surface of a substrate with reagents for in situ oligonucleotide synthesis, e.g., adapted for contacting a polynucleotide array with hybridization reagents.

In certain embodiments, the flow cell may comprises at least 10 inlet ports and at least 10 outlet ports.

In certain embodiments, the inlet and outlet ports are aligned along opposing sides of said flow cell.

An apparatus for in situ fabrication of a chemical array, comprising: a) a pulse-jet fluid deposition device for depositing chemical monomers onto a planar surface of a substrate; and b) a flow cell for contacting the surface with a liquid reagent, the flow cell comprising: i) a chamber comprising: i. a set of inlet ports and ii. a set of outlet ports; ii) a first manifold in fluid communication with the inlet ports; and iii) a second manifold in fluid communication with the outlet ports, wherein a plurality of the set of inlet ports and/or the set of outlet ports is independently controllable, is also provided.

In certain embodiments, the apparatus may be adapted so that a substrate may repeatedly placed in said pulse-jet fluid deposition device and said flow cell to produce the chemical array.

In certain embodiments, the apparatus may further comprising a transfer device for transferring said substrate from the pulse-jet fluid deposition device to the flow cell.

In certain embodiments, the chemical array may be a polynucleotide array and the chemical monomers may be nucleotide monomers.

A method of using a subject flow-cell, comprising: introducing a liquid into the flow cell through a first independently controllable inlet port of the plurality of inlet ports; and removing the liquid from the flow cell through a first independently controllable outlet port of the plurality of outlet ports, is also provided.

In certain embodiments, this method may further comprise terminating liquid flow through the first independently controllable inlet port.

In certain embodiments, this method may further comprise terminating liquid flow through the first independently controllable outlet port.

In particular embodiments, the method may include altering which inlet ports are used to introduce the liquid.

In particular embodiments, the method may include altering which outlet ports are used to remove the liquid.

In certain embodiments, the method may be for fabricating a chemical array and the method may include: depositing chemical monomers onto a planar surface of a substrate to generate a patterned surface; and contacting the patterned surface with the liquid in said flow cell.

In certain embodiments, the method may be for hybridizing a chemical array and may include: placing the chemical array in the flow cell; and introducing hybridization reagents into the flow cell via at least one of said inlet ports so that the hybridization reagent contacts the chemical array.

An array made using the method of is also provided.

A polynucleotide array comprising at least 100 polynucleotide features, wherein at least 50% of the polynucleotides within the features are full length, is also provided.

A method of sample analysis comprising: contacting a sample with a subject array and detecting binding of molecules in the sample on the array, is also provided.

### BRIEF DESCRIPTION OF THE FIGURES

FIGS. 1, 2 and 3 illustrate an exemplary substrate carrying a chemical array.

FIG. 4 schematically illustrates several features of one embodiment of the invention.

FIG. 5 schematically illustrates several features of a first embodiment of the instant methods.

FIG. 6 schematically illustrates an apparatus for array fabrication in accordance with the invention.

FIG. 7 schematically illustrates several features of a second embodiment of the instant methods.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer containing amino acid and/or nucleotide monomers, regardless of its source. A biopolymer may be naturally-occurring, obtained from a cell-based recombinant expression system, or synthetic. The term “biopolymer” refers to polypeptides and polynucleotides and includes compounds containing amino acids, nucleotides, or a mixture thereof.

The terms “polypeptide” and “protein” are used interchangeably throughout the application and mean at least two covalently attached amino acids. A polypeptide may be made up of naturally occurring amino acids and peptide bonds, synthetic peptidomimetic structures, or a mixture thereof. Thus “amino acid”, or “peptide residue”, as used herein encompasses both naturally occurring and synthetic amino acids and includes optical isomers of naturally occurring (genetically encodable) amino acids, as well as analogs thereof. For example, homo-phenylalanine, citrulline and noreleucine are considered amino acids for the purposes of the invention. The side chains may be in either the D- or the L- configuration. The term “amino acid” encompasses α- and β-amino acids.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids.

“Peptides” are generally greater than 2 amino acids, greater than 4 amino acids, greater than about 10 amino acids, greater than about 20 amino acids, usually up to about 10, 20, 30, 40 or 50 amino acids. In certain embodiments, peptides are between, 3 and 5 or 5 and 30 amino acids in length. In certain embodiments, a peptide may be three or four amino acids in length.

The term “fusion protein” or grammatical equivalents thereof is meant a protein composed of a plurality of polypeptide components that while typically unjoined in their native state, typically are joined by their respective amino and carboxyl termini through a peptide linkage to form a single continuous polypeptide. Fusion proteins may be a combination of two, three or even four or more different proteins. The term polypeptide includes fusion proteins, including, but not limited to, a fusion of two or more heterologous amino acid sequences, a fusion of a polypeptide with: a heterologous targeting sequence, a linker, an immunologically tag, a detectable fusion partner, such as a fluorescent protein, β-galactosidase, luciferase, etc., and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular and may contain modifications in the backbone to increase stability and half life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”).

A “polynucleotide” may be of any length, e.g., may contain 10-100 nucleotides or more than 200 nucleotides.

The term “oligonucleotide” as used herein denotes a single stranded polynucleotide of from 2 to 200 nucleotides in length. Oligonucleotides are usually synthetic and, in certain embodiments, are from 60-80 nucleotides in length. As used herein, the terms “oligonucleotide” and “polynucleotide” are generally used interchangeably unless context dictates otherwise.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

The term “monomer” as used herein refers to a chemical entity that can be covalently linked to one or more other such entities to form an polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art synthesis of nucleic acids of this type utilizes an initial substrate-bound monomer that is generally used as a building-block in a multi-step synthesis procedure to form a complete nucleic acid.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base. In the practice of the instant invention, oligomers will generally comprise about 2-60 monomers, preferably about 10-60, more preferably about 50-60 monomers.

The term “sample” as used herein relates to a material or mixture of materials, typically, although not necessarily, in,fluid form, containing one or more components of interest.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “protection” and “deprotection” as used herein relate, respectively, to the addition and removal of chemical protecting groups using conventional materials and techniques within the skill of the art and/or described in the pertinent literature; for example, reference may be had to Greene et al., Protective Groups in Organic Synthesis, 2nd Ed., New York: John Wiley & Sons, 1991. Protecting groups prevent the site to which they are attached from participating in the chemical reaction to be carried out.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

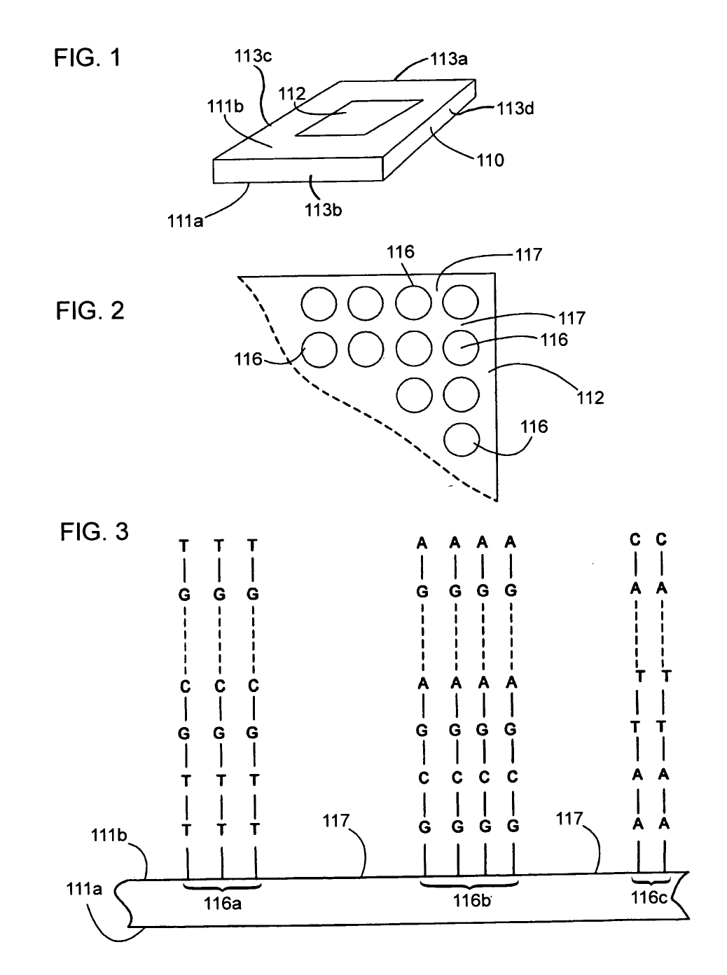

An exemplary array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes which are bound to the substrate at the various regions. However, either of the “target” or “probe” may be the one which is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information references transmitting the data representing that information as electrical signals over a suitable communication channel (e.g., a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports).

It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

A “plurality” is at least 2, e.g., 2, at least about 5, at least about 10, at least about 20, at least about 50, at least about 100, or at least about 1000. The phrases “a plurality of” and “multiple” are used interchangeably. A plurality of elements contains at least a first element and a second element.

A “set” of elements may include one or more elements.

A “manifold” contains a chamber and several outlets.

### DESCRIPTION

In one embodiment, the invention provides a flow cell having a plurality of independently controllable inlet and/or outlet ports. In general terms, the subject flow cell contains: a) a chamber having a plurality of independently controllable inlet ports and/or a plurality of independently controllable outlet ports; b) a first manifold in fluid communication with the inlet ports; and c) a second manifold in fluid communication with the outlet ports. In one embodiment, a subject flow cell may be employed in conjunction with a pulse-jet fluid deposition device as part of an apparatus for fabrication of a chemical array. Exemplary methods of using a subject flow cell to control liquid flow over a planar substrate are provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, the subject invention provides a flow cell containing independently controllable inlet and independently controllable outlet ports. In further describing the invention, the subject flow cell and methods by which liquid flow may be used to control liquid flow will be described first, followed by a description of an array fabrication apparatus containing a subject flow cell. Exemplary uses of a subject flow cell are then discussed.

### Flow Cells

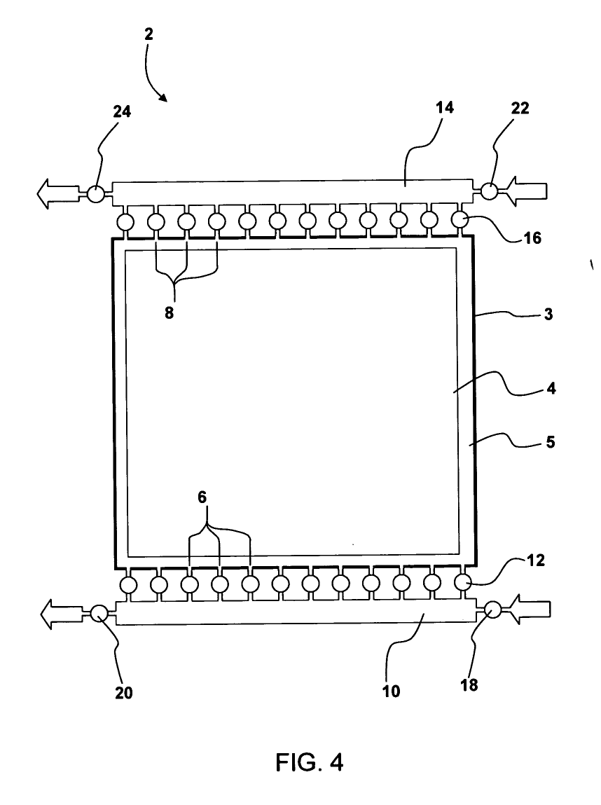

Many of the general features of a subject flow cell according to an aspect of the invention are schematically illustrated in FIG. 4. In general terms and as illustrated in FIG. 4, a subject flow cell 2 contains a housing 3 that defines a chamber 5 that is dimensioned for receiving a substrate 4 having a surface (e.g., such as a planar surface) that is to be contacted with a liquid. The flow cell allows a liquid to pass through the chamber and contact a substrate disposed within the chamber. In certain embodiments, the substrate may be positioned in the chamber using a substrate holder. The housing further contains a plurality of inlet ports 6 for introducing liquid into the chamber and a plurality of outlet ports 8 for removing liquid from the chamber (e.g., for “venting” the chamber). Each of the inlet ports 6 is independently controllable in that it is connected to a first manifold 10 by via a first independently actuatable valve 12. The flow cell contains a plurality of first independently actuatable valves, each associated with an inlet port. The first independently actuatable valves are independently actuatable in that one of more of the valves may be opened or closed independently of the remainder of the valves. Each of the outlet ports 8 is independently controllable in that it is connected to a second manifold 6 by via a second independently actuatable valve 16. The flow cell contains a plurality of second independently actuatable valves, each associated with a valve. The second independently actuatable valves are independently actuatable in that one of more of the valves may be opened or closed independently of the remainder of the valves.

In other words, the subject flow cell contains a chamber 5 and a first manifold 10 that are fluidically connected via a plurality of inlet ports that are each associated with an independently actuatable valve 12. Liquid flow from the first manifold 10 to the chamber 5 may be through any single inlet port or any number of inlet ports in any combination, including all inlet ports. The inlet port(s) employed to introduce liquid into the chamber 5 may change over any time period. In additional embodiments, the subject flow cell contains a chamber 5 and a second manifold 14 that is fluidically connected to chamber 5 via a plurality of outlet ports that are each associated with an independently actuatable valve 16. Liquid flow from the chamber 5 to the second manifold 14 may be through any single outlet port or any number of outlet ports in any combination, including all outlet ports. The outlet port(s) employed to introduce liquid into the chamber 5 may change over any time period.

In one embodiment, the inlet ports 6 are positioned in a line along one side of chamber 5 and the outlet ports 8 are positioned in a line along the opposing side of chamber 5 (i.e., such that a liquid that has entered chamber 5 through inlet ports 10 will pass over and make contact with a substrate disposed in chamber 5 prior to exiting the chamber though outlet ports 8. In certain embodiments, a subject flow cell may contain at least 2, at least about 4, at least about 8, at least about 12, at least about 20, at least about 50, at least about 100, up to about 1,000 or more inlet ports. In certain embodiments, a subject flow cell may contain at least 2, at least about 4, at least about 8, at least about 12, at least about 20, at least about 50, at least about 100, up to about 1,000 or more outlet ports. A subject flow cell need not contain the same number of inlet ports and outlet ports.

Each inlet port or outlet port may be connected to a manifold via a separate fluid transport element, e.g., tubing or the like, that contains a flow regulation device, e.g., a valve. Inlet ports 6 and outlet ports 8 may be of any suitable shape (e.g., circular, square, hexagonal, irregular, oval or elongated, etc.) and size (e.g., in the range of about 100 μm to about 5 mm or about 500 μm to about 2 mm in diameter) and may be spaced apart at regular or irregular intervals. In one embodiment and as shown in FIG. 4, the inlet ports and outlet ports may be equidistantly spaced along opposing sides of chamber 5.

In a further embodiment, liquid entry into and exit from the first manifold 10 and second manifold 14 is also controllable. In this embodiment, liquid flow through the entrance of the first manifold 10 is controllable via valve 18, and liquid flow through the exit of first manifold 10 is controllable via valve 20. Valves 18 and 22 are independently actuatable in that either of the valves may be opened or closed independently of the other valve. Likewise, liquid flow through the entrance of the second manifold 14 is controllable via valve 22, and liquid flow through the exit of second manifold 14 is controllable via valve 24. Valves 22 and 24 are independently actuatable in that either of the valves may be opened or closed independently of the other valve.

Since liquid flow into and out of the first manifold 10 and the second manifold 14 is controllable, the direction of liquid flow through chamber 5 may be in any direction, i.e., from the inlet ports 6 to the outlet ports 8 or from the outlet ports 8 to the inlet ports 6. The direction of flow of a liquid through a subject flow cell may be reversed at any time during its use.

Valves 12, 16, 18, 20, 22 and 24 may be any suitable type of valve, including valves that may be actuated between open and closed positions manually, non-manually, hydraulically, pneumatically or electrically e.g., piezoelectrically or electromagnetically. In one embodiment, the valves are solenoid valves, which are actuated between open and closed positions by a solenoid.

The overall dimensions of the chamber of the subject flow cell may vary and are dependent on the dimensions of the substrate that is to be placed therein. In certain embodiments, the substrate may be one on which an array of compounds is synthesized. In these embodiments, the substrate may be about 1.5. to about 5 inches in length and about 0.5 to about 3 inches in width. The substrate may be about 0.1 to about 5 mm, e.g., about 0.5 to about 2 mm, in thickness. A standard size microscope slide is about 3 inches in length and about 1 inch in width. In certain embodiments, multiple arrays of chemical compounds may be synthesized on the substrate, which is then diced, i.e., cut, into single array substrates. In this embodiment, the substrate may be about 5 to about 8 inches in length and about 5 to about 8 inches in width so that the substrate may be diced into multiple single array substrates having the aforementioned dimensions. The thickness of the support is the same as that described above. In a specific embodiment by way of illustration and not limitation, a wafer that is 6^{5/8} inches by 6 inches may be employed and diced into one inch by 3 inch slides.

Representative flow cells are about 6.5 inches wide by about 6 inches tall in the plane of the flow cell. However, in certain embodiments, these dimensions can range from about 1 cm square to about 1 meter square.

Flow rate through any one given port may vary greatly. In certain embodiments, the flow rate may be in the range of 0.1 μl/sec to 10.0 ml/sec, e.g., about 1 μl/sec to about 1 ml/sec, although flow rates outside of this range may be used in certain circumstances.

The housing of a subject flow cell is generally constructed to permit access into the chamber therein. The flow cell may contain an opening that is sealable to prevent fluid leakage after the substrate is placed therein. Such seals may comprise a flexible material that is sufficiently flexible or compressible to form a fluid tight seal that can be maintained under increased pressures encountered in the use of the device. The flexible member may be, for example, rubber, flexible plastic, flexible resins, and the like and combinations thereof. In any event, the flexible material should be substantially inert with respect to the fluids introduced into the device and must not interfere with the reactions that occur within the device. The flexible member is usually a gasket and may be in any shape such as, for example, circular, oval, rectangular, and the like. In certain embodiments, the flexible member is in the form of an O-ring.

In other embodiments, the housing of the flow cell may be conveniently constructed in two parts, which may be referred to generally as top and bottom elements. These two elements may be sealably engaged during contact of a substrate to a liquid, and separated at other times to permit the substrate to be placed into and removed from the chamber of the flow cell. In certain embodiments, the top element may be adapted to be moved with respect to the bottom element.

The inlets of the flow cell is usually in fluid communication with an element that controls the flow of fluid into the flow cell such as, for example, a manifold, a valve, and the like or any combination thereof. This element in turn is in fluid communication with one or more fluid reagent dispensing stations. In this way the substrate may be contacted with multiple different liquids (e.g., liquids containing different chemical reagents) in the flow cell.

In one embodiment, the fluid dispensing stations are affixed to a base plate or platform to which the flow cells are mounted. Any fluid dispensing station may be employed to dispense a fluid such as water, aqueous media, organic solvents, ionic liquids or the like. The fluid dispensing station may comprises a pump for moving fluid and may also comprise a valve assembly and a manifold as well as a fluid delivery element (e.g., a syringe or a valved device) for delivering predetermined quantities of fluid to the flow cell. The fluids may be dispensed by pumping from the dispensing station. In this regard, any standard pumping technique for pumping fluids may be employed in the present apparatus. For example, pumping may be by means of a peristaltic pump, a pressurized fluid bed, a positive displacement pump, e.g., a syringe pump, and the like.

A subject flow cell may be made of any of a wide variety of materials, e.g., metal, plastic or dielectric, for example, and may be coated in order to provide particular surface properties. A subject flow cell may contains a planar element, e.g., a lid, base, wall or internal plate or the like, that contains a planar surface that lies parallel to the planar surface of a substrate that is disposed within the chamber of the flow cell.

Methods

The above-described multi-port flow cell may be employed in a variety of different methods. In one embodiment, these methods involve placing a substrate in a subject flow cell, introducing a liquid into the flow cell through at least one independently controllable inlet port, and removing the liquid from the flow cell through at least one of the independently controllable outlet ports. In other embodiments, the methods may further involve terminating liquid flow through the independently controllable inlet port and/or terminating liquid flow through the independently controllable outlet port.

In certain embodiments, the methods involve introducing a first liquid by a first set of inlet ports (where a set includes at least one and up to all of a plurality,. e.g., at least 2, at least about 4 or at least about 10 inlet ports, etc.), and then altering which inlet ports are employed to introduce the first liquid. In other words, in certain embodiments, the set of inlet ports employed to introduce liquid into a subject flow cell may be altered after that liquid has started to flow through the flow cell. Likewise, the set of outlet ports employed to remove liquid from a flow cell may be altered after that liquid has started to flow through the flow cell.

In certain embodiments, the sets of inlet ports and outlet ports used in moving liquid through a subject flow cell may be altered without terminating the flow of liquid through the flow cell. In other words, the sets of inlet and outlet ports used in a subject flow cell may be altered during the continuous flow of a liquid through the flow cell. In altering the sets of inlet and outlet ports employed in a subject flow cell, the original and altered sets of ports may or may not overlap (i.e., may or may not contain a port in common).

The sets of inlet and outlet ports used at any one time may contain ports that are adjacent to one another, non-adjacent to one another, or may contain ports that are adjacent and non-adjacent. Further, there is no requirement that the outlet ports employed at any one time are related in number or position to the inlet ports employed at that time. The ports employed may be chosen at random, or pre-determined, for example.

The sets of inlet and outlet ports employed may be changed at any time. In particular times, the sets of inlet and outlet ports that may be employed may be changed multiple times (e.g., at least twice, at least about 10, or in certain embodiments, at least about 50, at least about 100 or at least about 1,000 times or more) as a particular liquid flows though the flow cell. The changes may be made at random times, selected times (e.g., as a user monitors conditions within the flow cell), pre-determined times or regular intervals, for example. In representative embodiments, the changes may be made every second, every minute, every 5 minutes or every hour or more, etc.

In particular embodiments, the changes are at regular intervals and employ pre-determined sets of inlet ports and outlet ports in order to provide a particular flow pattern of a liquid across the substrate.

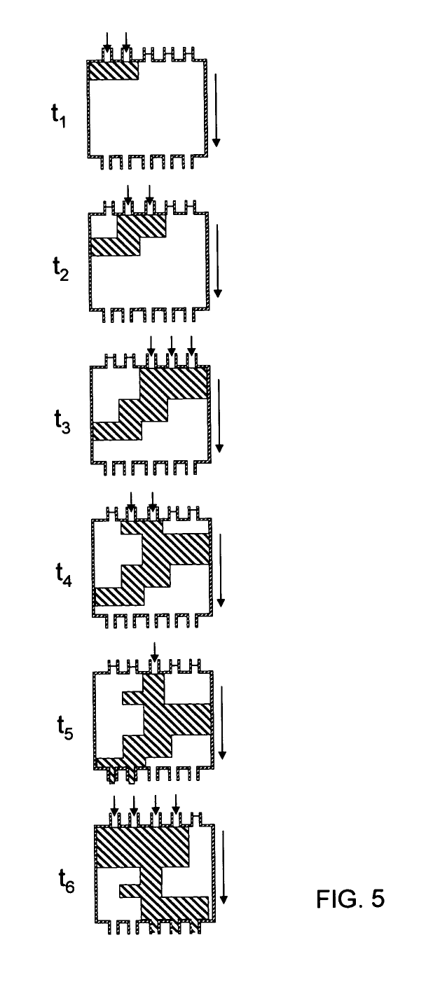

An embodiment of the instant method is schematically illustrated in FIG. 5, showing a subject flow cell at six arbitrary times (t₁, t₂, t₃, t₄, t₅ and t₆) during use of the flow cell. In this embodiment, the flow cell contains 5 inlet ports (at the top of each flow cell) that are independently controllable and 5 outlet ports (at the bottom of each flow cell) that also open independently controllable. In this embodiment, the 5 outlet ports are all in the “open” position. As illustrated in FIG. 5, any single inlet port or combination of inlet ports may be used to introduce liquid (indicated by hatching) into the flow cell. The outlet ports can be controlled in a similar manner to the inlet ports. It is noted that FIG. 5 is a schematic illustration. In actual use of a subject flow cell, the boundaries of a liquid flowing through the cell may not be sharp and straight as illustrated, and the flow of a liquid through a cell is not completely linear. Further, the direction of liquid in a flow cell need not be “from top to bottom”. As well understood in the art, the direction of liquid flow across a substrate in a flow cell may be parallel to the direction of earth's gravitational pull, anti-parallel to the direction of earth's gravitational pull, or at any angle in between those directions (e.g., at 90° the direction of gravitational pull). In exemplary embodiments, in use of a subject flow cell, the substrate may be upright, lying down, or at any angle between upright and lying down, relative to the earth's surface. The direction of liquid flow through a subject flow cell may be from any end of the substrate to the other.

In use of a subject flow cell, areas of a substrate that would otherwise be thought of “dead spots” can be contacted with a liquid by directing liquid to that spot using a particular combination of inlet and outlet ports, or by varying the set of inlet and outlet ports used. Further, by varying the inlet and outlet ports used, different liquids may be mixed or folded together in new ways. In one embodiment of interest, the subject flow cell may be used to deliberately create so called “dead-spots”, allowing the study of the effect of a particular liquid on the substrate.

In a particular embodiment noted above, the device is configured so that the direction of flow of a liquid through a subject flow cell may be reversed at any time during use of the device. In one embodiment, the subject flow cell may be employed in a method in which a first liquid is introduced into the flow cell via a first set of ports on one side of a flow cell. After a period of time, flow through the first set of ports is terminated, and a second liquid is introduced into the flow cell via a second set of ports on the opposing side of the flow cell. In this embodiment, the first and second liquids may be the same liquid or different liquids (i.e., may contain the same or different components). This embodiment can be employed so that exposed areas of a substrate are uniformly and thoroughly contacted with a liquid, e.g., for washing the substrate with a wash solution. In this case, the solution may be introduced into the flow cell, and moved back and forth across the substrate to provide uniform contact of the liquid with the substrate.

### Apparatus Containing a Subject Flow Cell

The above-described subject flow cell may be particularly employed in any method or system in which it is desirable or necessary to contact a surface, e.g., a planar surface, with a liquid. In certain embodiments, therefore, the subject flow cell may be used in the fabrication, cleaning or use of a variety of planar substrates. For example, a subject flow cell may be employed with a chemical array (e.g., in the fabrication or hybridization of a polynucleotide or polypeptide array), a semiconductor wafer (e.g., any semiconductor wafer such as a chip employed in a programmable logic device, memory, a chipsets or a networking integrated circuit), or a micro-fabricated optical component (e.g., a micro-patterned lens, diffuser or filter), each of which contain a planar surface.

In one representative embodiment described in greater detail below in order to exemplify but not limit the invention, the subject flow cell may be employed in conjunction with a pulse-jet fluid deposition device in an apparatus for fabricating a chemical array. This apparatus generally contains: a pulse-jet fluid deposition device for depositing chemical monomers in a pattern onto a planar surface of a substrate to produce a patterned surface; and a subject flow cell adapted for contacting the patterned surface with a liquid reagent. In certain embodiments, the subject apparatus may be adapted so that a substrate may be repeatedly positioned in the pulse-jet fluid deposition device and the subject flow cell to produce the chemical array. In one embodiment, the apparatus may further contain a transfer device for transferring the substrate from the pulse-jet fluid deposition device to a subject flow cell.

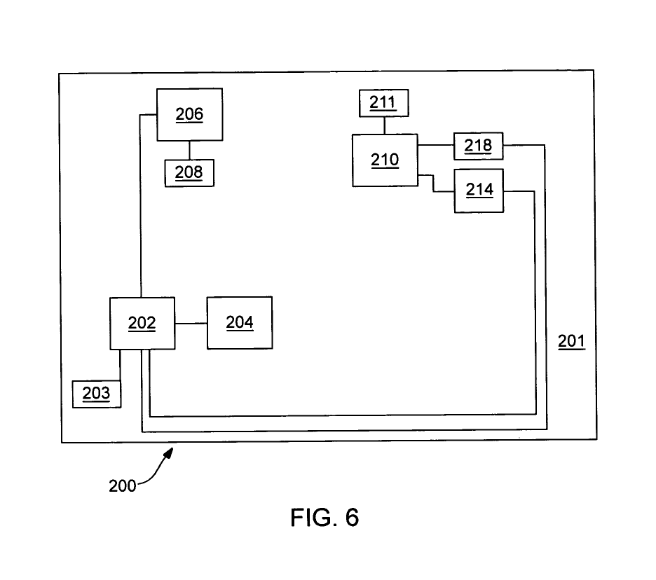

An embodiment of an array fabrication apparatus that contains a subject flow cell is schematically depicted in FIG. 6. Subject apparatus 200 comprises platform 201 on which the components of the apparatus are mounted. Apparatus 200 comprises computer 202, with which various components of the apparatus are in communication. Video display 203 is in communication with computer 202. Apparatus 200 further comprises print chamber 204, which is controlled by computer 202. The type of print chamber 204 used depends on the nature of the printing technique employed to add monomers to a growing polymer chain or to deposit polymers on an array substrate. Such printing techniques include, by way of illustration and not limitation, pulse-jet deposition, and so forth. Transfer robot 206 is also controlled by computer 202 and comprises a robot arm 208 that moves a support to be printed from print chamber 204 to a subject flow cell 210. In one embodiment robot arm 208 introduces a support into print chamber 204 horizontally for printing on a surface of the support and introduces the support into a flow cell vertically. Flow cell 210 is in communication with program logic controller 214, which is controlled by main computer 202. Flow cell 210 is in communication with fluid dispensing station 211 and flow sensor and level indicator 218, which-are controlled by main computer 202.

The apparatus of the invention further includes appropriate electrical and mechanical architecture and electrical connections, wiring and devices such as timers, clocks, and so forth for operating the various elements of the apparatus. Such architecture is familiar to those skilled in the art and will not be discussed in more detail herein.

The apparatus may be controlled with the aid of a computer. For example, an IBM® compatible personal computer (PC) may be utilized. The computer is driven by software specific to the methods described herein. Computer hardware capable of assisting in the operation of the methods in accordance with the present invention involves in certain embodiments a system with at least the following specifications: Pentium® processor or better with a clock speed of at least 100 MHz, at least 32 megabytes of random access memory (RAM) and at least 80 megabytes of virtual memory, running under either the Windows 95 or Windows NT 4.0 operating system (or successor thereof). Software that may be used to carry out the methods may be, for example, Microsoft Excel or Microsoft Access, suitably extended via user-written functions and templates, and linked when necessary to stand-alone programs. Examples of software or computer programs used in assisting in conducting the present methods may be written, preferably, in Visual BASIC, Labview, FORTRAN and C⁺⁺. It should be understood that the above computer information and the software used herein are by way of example and not limitation. The present methods may be adapted to other computers and software. Other languages that may be used include, for example, PASCAL, PERL or assembly language.

A computer program may be utilized to carry out the above method steps. The computer program provides for the independent control of the inlet and outlet ports by controlling (i.e., independently opening and closing) the valves that are associated with those ports. The computer program further may provide for moving the support to and from a station for monomer addition at a predetermined point in the aforementioned method.

### Utility

As mentioned above, the subject flow cell may be employed with a wide variety of substrates having a patterned surface. In exemplary embodiments, a subject flow cell may be employed in fabrication of a chemical array (e.g., a polynucleotide or polypeptide array) or in a method of sample analysis that employs a chemical array.

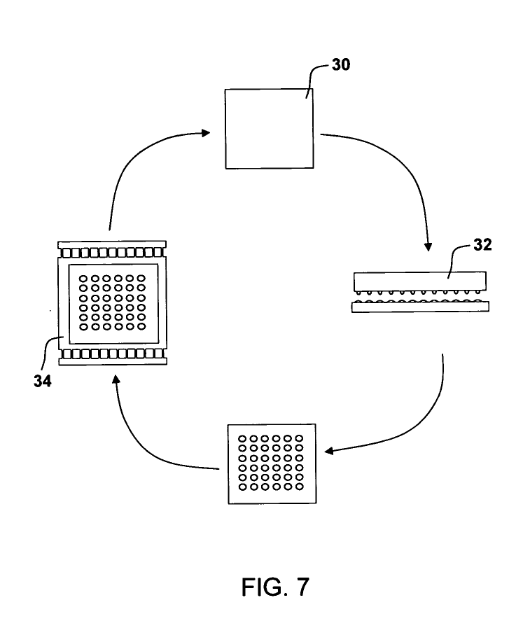

In certain chemical array fabrication methods and as illustrated in FIG. 7, a substrate 30 is placed in a pulse-jet deposition device 32, and the device deposits chemical monomers (e.g., nucleotides or amino acids) in a pattern onto the surface of the substrate. The chemical monomers link to growing polymer chains that are attached to the surface of the substrate. After deposition, the substrate is removed from the device and placed in a subject flow cell 34, where the substrate is contacted with a liquid containing reactants that modify the most recently added chemical monomers and prepare the growing polymer chain for addition of a further chemical monomer. After contact with the reactants, the substrate is removed from the flow cell and re-positioned in the pulse-jet deposition device. The substrate is repeatedly placed in the pulse-jet deposition device and flow cell until the desired polymers have been made.

Accordingly, in one embodiment, the invention may be used in the synthesis of a polypeptide array. The synthesis of polypeptides involves the sequential addition of amino acids to a growing peptide chain. This approach comprises attaching an amino acid to the functionalized surface of the support. In one approach the synthesis involves sequential addition of carboxyl-protected amino acids to a growing peptide chain with each additional amino acid in the sequence similarly protected and coupled to the terminal amino acid of the oligopeptide under conditions suitable for forming an amide linkage. Such conditions are well known to the skilled artisan. See, for example, Merrifield, B. (1986), Solid Phase Synthesis, Sciences 232, 341-347. After polypeptide synthesis is complete, acid is used to remove the remaining terminal protecting groups. In accordance with the present invention, certain steps involved in the addition of an amino acid may be carried out in a subject flow cell. Such steps may involve, among others, washing of the surface, protection and deprotection of certain functionalities on the surface, oxidation or reduction of functionalities on the surface, and so forth.

In a second embodiment, the invention may be used in the synthesis of a nucleic acid array, e.g., an oligonucleotide array. The synthesis of arrays of nucleic acids on the surface of a support in certain approaches, e.g., in situ fabrication protocols, involves attaching an initial nucleoside or nucleotide to a functionalized surface. In one approach the surface is reacted with nucleosides or nucleotides that are also functionalized for reaction with the groups on the surface of the support. Methods for introducing appropriate amine specific or alcohol specific reactive functional groups into a nucleoside or nucleotide include, by way of example, addition of a spacer amine containing phosphoramidites, addition on the base of alkynes or alkenes using palladium mediated coupling, addition of spacer amine containing activated carbonyl esters, addition of boron conjugates, formation of Schiff bases.

After the introduction of the nucleoside or nucleotide onto the surface, the attached nucleotide may be used to construct the polynucleotide by means well known in the art. For example, in the synthesis of arrays of oligonucleotides, nucleoside monomers are generally employed. In this embodiment an array of the above compounds is attached to the surface and each compound is reacted to attach a nucleoside. Nucleoside monomers are used to form the polynucleotides usually by phosphate coupling, either direct phosphate coupling or coupling using a phosphate precursor such as a phosphite coupling. Such coupling thus includes the use of amidite (phosphoramidite), phosphodiester, phosphotriester, H-phosphonate, phosphite halide, and the like coupling.

One exemplary coupling method is phosphoramidite coupling, which is a phosphite coupling. In using this coupling method, after the phosphite coupling is complete, the resulting phosphite is oxidized to a phosphate. Oxidation can be effected with iodine to give phosphates or with sulfur to give phosphorothioates. The phosphoramidites are dissolved in anhydrous acetonitrile to give a solution having a given ratio of amidite concentrations. The mixture of known chemically compatible monomers is reacted to a solid support, or further along, may be reacted to a growing chain of monomer units. In one particular example, the terminal 5′-hydroxyl group is caused to react with a deoxyribonucleoside-3′-O—(N,N-diisopropylamino)phosphoramidite protected at the 5′-position with dimethoxytrityl or the like. The 5′ protecting group is removed after the coupling reaction, and the procedure is repeated with additional protected nucleotides until synthesis of the desired polynucleotide is complete. For a more detailed discussion of the chemistry involved in the above synthetic approaches, see, for example, U.S. Pat. No. 5,436,327 at column 2, line 34, to column 4, line 36, which is incorporated herein by reference in its entirety.

Certain steps in the above-described method may be performed in a subject flow cell, e.g., washing the surface of the support prior to or after a reaction, oxidation of substances such as oxidation of a phosphite group to a phosphate group, removal of protecting groups, blocking of sites to prevent reaction at such site, capping of sites that did not react with a phosphoramidite reagent, deblocking, and so forth. In addition, under certain circumstances other reactions may be carried out in a subject flow cell such as, for example, phosphoramidite monomer addition, modified phosphoramidite addition, other monomer additions, addition of a polymer chain to a surface for linking to monomers, and so forth.

For in situ fabrication methods, multiple different reagent droplets are deposited by pulse jet or other means at a given target location in order to form the final feature (hence a probe of the feature is synthesized on the array substrate). The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents. The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence can be considered as multiple ones of the following attachment cycle at each feature to be formed: (a) coupling an activated selected nucleoside (a monomeric unit) through a phosphite linkage to a functionalized support in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The coupling can be performed by depositing drops of an activator and phosphoramidite at the specific desired feature locations for the array. Capping, oxidation and deprotection can be accomplished by contacting the entire substrate (“flooding”) with a layer of the appropriate reagent in a subject flow cell. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in another flooding procedure in a known manner. Conventionally, a single pulse jet or other dispenser is assigned to deposit a single monomeric unit.

The foregoing chemistry of the synthesis of polynucleotides is described in detail, for example, in Caruthers, Science 230: 281-285, 1985; Itakura, et al., Ann. Rev. Biochem. 53:323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643 and European patent application, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in Southern, E. M., Maskos, U. and Elder, J. K., Genomics, 13, 1007-1017, 1992.

In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

In another embodiment, the subject flow cell and method may be employed in an array binding protocol. In these embodiments, an array may be placed in a subject flow cell, contacted with binding reagent, e.g., hybridization reagent, optionally washed, and then drained using the subject methods. The drained array may then be read to provide results.

Protocols for carrying out such assays are well known to those of skill in the art and need not be described in great detail here. Generally, a sample is contacted with an array in a subject flow cell under conditions sufficient for analytes in the sample to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, comparative genomic hybridization assays, location analysis assays (e.g., ChIP-chip) and the like. Patents and patent applications describing methods of using arrays in various applications include: U.S. Pat. No. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array, the array is typically exposed to a sample in a subject flow cell (for example, a fluorescently labeled analyte, e.g., protein or nucleic acid-containing sample), and, after the array is washed and drained, the array then read. Reading of the array may be accomplished by illuminating the array and reading the location and intensity of resulting fluorescence at each feature of the array to detect any binding complexes on the surface of the array. For example, a scanner may be used for this purpose which is similar to the scanner available from Agilent Technologies, Palo Alto, Calif. Other suitable apparatus and methods are described in U.S. patent applications: Ser. No. 09/846125 “Reading Multi-Featured Arrays” by Dorsel et al.; and Ser. No. 09/430214 “Interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

Arrays

The arrays made using the above-described system contain at least two distinct biopolymers that differ by monomeric sequence immobilized on, e.g., covalently to, different and known locations on the substrate surface. In certain embodiments, each distinct biopolymer sequence of the array is typically present as a composition of multiple copies of the polymer on the substrate surface, e.g., as a spot on the surface of the substrate. The number of distinct biopolymer sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the polymeric sequences are not arranged in the form of distinct spots, but may be positioned on the surface such that there is substantially no space separating one polymer sequence/feature from another.

As indicated above, the arrays are arrays of nucleic acids, including oligonucleotides, polynucleotides, DNAs, RNAs, synthetic mimetics thereof, or arrays of polypeptides, including peptides, and the like.

An attribute of the subject arrays, which can result from the use of the flow cell and methods described above, is that each feature (i.e., collection of probes at a discrete spot) of the arrays is highly uniform in terms of its composition, since an array is uniformly contacted with liquid in the flow cell. As such, the proportion of full-length sequence within each feature is generally higher (e.g., at least about 5% higher, often at least about 10% higher, such as at least about 25%, 50%, 90%, 95%, 99% or 100% higher) as compared to arrays produced using analogous protocols that do not include the method and system described above. As a result, the signal detected from these arrays is higher (e.g., at least about 5% higher, often at least about 10% higher, such as at least about 25%, 50% or 100% higher) than signals detected from arrays made using analogous protocols that do no include the inventive method and system described above All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A flow cell comprising:
a) a chamber comprising:
i. a set of inlet ports and
ii. a set of outlet ports;
b) a first manifold in fluid communication with said inlet ports; and
c) a second manifold in fluid communication with said outlet ports,
wherein a plurality of said set of inlet ports and/or said set of outlet ports is independently controllable.

2. The flow cell of claim 1, wherein said inlet ports and said outlet ports are connected to said first and second manifolds via independently actuatable valves.

3. The flow cell of claim 1, wherein said inlet ports and said outlet ports are connected to said first and second manifolds via independent solenoid valves.

4. The flow cell of claim 1, wherein flow into and out of said first manifold is independently controllable.

5. The flow cell of claim 1, wherein flow into and out of said second manifold is independently controllable.

6. The flow cell of claim 1, wherein said flow cell is adapted for contacting a surface of a substrate with reagents for in situ oligonucleotide synthesis.

7. The flow cell of claim 1, wherein said flow cell is adapted for contacting a polynucleotide array with hybridization reagents.

8. The flow cell of claim 1, wherein said flow cell comprises at least 10 inlet ports and at least 10 outlet ports.

9. The flow cell of claim 1, wherein said inlet and outlet ports are aligned along opposing sides of said flow cell.

10. An apparatus for in situ fabrication of a chemical array, comprising:
a) a pulse-jet fluid deposition device for depositing chemical monomers onto a planar surface of a substrate; and
b) a flow cell for contacting said surface with a liquid reagent, said flow cell comprising:
i) a chamber comprising:
i. a set of inlet ports and
ii. a set of outlet ports;
ii) a first manifold in fluid communication with said inlet ports; and
iii) a second manifold in fluid communication with said outlet ports,
wherein a plurality of said set of inlet ports and/or said set of outlet ports is independently controllable.

11. The apparatus of claim 10, wherein said apparatus is adapted so that a substrate is repeatedly placed in said pulse-jet fluid deposition device and said flow cell to produce said chemical array.

12. The apparatus of claim 10, further comprising a transfer device for transferring said substrate from said pulse-jet fluid deposition device to said flow cell.

13. The apparatus of claim 10, wherein said chemical array is a polynucleotide array and said chemical monomers are nucleotide monomers.

14. A method of using a flow-cell of claim 1, comprising:
introducing a liquid into said flow cell through a first independently controllable inlet port of said plurality of inlet ports;
removing said liquid from said flow cell through a first independently controllable outlet port of said plurality of outlet ports.

15. The method of claim 14, further comprising terminating liquid flow through said first independently controllable inlet port.

16. The method of claim 15, further comprising terminating liquid flow through said first independently controllable outlet port.

17. The method of claim 15, wherein said method includes altering which inlet ports are used to introduce said liquid.

18. The method of claim 16, wherein said method includes altering which outlet ports are used to remove said liquid.

19. The method of claim 16, wherein said method is a method for fabricating a chemical array and said method includes:
depositing chemical monomers onto a planar surface of a substrate to generate a patterned surface;
contacting said patterned surface with said liquid in said flow cell.

20. The method of claim 15, wherein said method is a method of hybridizing a chemical array and said method includes:
placing said chemical array in said flow cell; and
introducing hybridization reagents into said flow cell via at least one of said inlet ports so that said hybridization reagent contacts said chemical array.

21. An array made using the method of claim 19.

22. An array comprising at least 100 polynucleotide features, wherein at least 50% of the polynucleotides within said features are full length.

23. A method of sample analysis comprising:
contacting a sample with an array of claim 21 and detecting binding of molecules in the sample on the array.
